(19) United States
(12) Patent Application Publication
(10) Pub. No.: US 2021/0346103 A1
(43) Pub. Date: Nov. 11, 2021

(54) SUPPORT FOR A MEDICAL INTERVENTION

(71) Applicant: Siemens Healthcare GmbH, Erlangen (DE)

(72) Inventor: Armin Stranjak, Uttenreuth (DE)

(21) Appl. No.: 17/315,247

(22) Filed: May 7, 2021

(30) Foreign Application Priority Data
May 8, 2020 (DE) 10 2020 205 804.0

(51) Int. Cl. A61B 34/00; A61B 34/20; H04R 1/10; G06F 3/16

(52) U.S. Cl. CPC A61B 34/25; A61B 34/20; A61B 34/76; A61B 2090/374; H04R 1/1041; G06F 3/165; A61B 2034/2065; H04R 1/1008

(57) ABSTRACT

A system for supporting a medical intervention, a method for output of a feedback signal, and a computer program product are provided. The system includes a feedback device for output of a feedback signal to a user of an intervention apparatus, and a control device for controlling the feedback device. The control device is configured to receive an actual position of the intervention apparatus and a target position of the intervention apparatus. The control device is further configured to determine a deviation of the actual position from the target position and to control the output of the feedback signal by the feedback device as a function of the deviation. In this case, the feedback signal is a non-visual feedback signal.

This application claims the benefit of German Patent Application No. DE 10 2020 205 804.0, filed on May 8, 2020, which is hereby incorporated by reference in its entirety.

### BACKGROUND

The present embodiments relate to a system for supporting a medical intervention, to a method for output of a feedback signal, and to a computer program product.

Ablation, biopsy, and puncturing are among the methods used most frequently during a medical intervention, in which an intervention apparatus is introduced by a user (e.g., a doctor) into the body of a patient. The intervention apparatus may be a needle, for example. A tissue sample, which is subsequently analyzed, is removed with the aid of a biopsy needle, for example. In order to carry out such an intervention safely and efficiently, a continuous monitoring of the guidance of a needle up to the point at which the tissue sample is to be removed is to be provided. The expected path of the needle between the entry point (e.g., the skin surface) and the target part of the body is carefully planned before the intervention.

As soon as the path is defined, it is to be provided that the needle follows this predefined path. Since the needle is usually introduced manually and is controlled by a human hand, the position of the needle may easily deviate from the specified path and is therefore to be constantly monitored. For example, a magnetic resonance image is created with an imaging device (e.g., a magnetic resonance device), of which the imaging area includes the path, so that the needle is always visible in the magnetic resonance image when the needle is correctly inserted and the planned path is being correctly followed.

The user attempts during the intervention to always keep the needle on the path (e.g., and to make the needle visible on the magnetic resonance image) by adjusting the trajectory of the needle manually. In order to achieve this, the magnetic resonance image is usually displayed on a screen, and the user checks the progress of the penetration of the needle into the body of the patient by looking at the screen.

Usually in such cases, a slice that contains the planned path of the needle between entry point and target region is shown in real time. In such cases, it is possible for the needle to stray from the planned path and therefore no longer to be visible on the screen. In such a case, the user would have to attempt to bring the needle onto the path again. This is normally done by the user pulling the needle back and trying another route while attempting to see the needle again on the monitor. This method of operation may be error-prone, time-consuming, stressful, and uncomfortable for the patient.

Such a method requires a continuous visual observation of body and needle of the patient and of the screen display. This requires a constant focusing or defocusing of the eyes of the user. This too may be stressful and error-prone. Further, switching between the activities of introducing the needle and looking at the display slows down the process and increases user fatigue.

### SUMMARY AND DESCRIPTION

The scope of the present invention is defined solely by the appended claims and is not affected to any degree by the statements within this summary.

It would be desirable for the user to be able to concentrate attention more strongly on operating the intervention apparatus.

The present embodiments may obviate one or more of the drawbacks or limitations in the related art. For example, a system for supporting a medical intervention that allows the user to be able to concentrate attention more strongly on operating the intervention apparatus is provided. The system includes a feedback device for output of a feedback signal to a user of the intervention apparatus and a control device for controlling the feedback device. In this system, the control device is configured to receive an actual position of the intervention apparatus and a target position of the intervention apparatus, to determine a deviation of the actual position from the target position, and to control the output of the feedback signal by the feedback device as a function of the deviation. The feedback signal is, for example, a non-visual feedback signal.

The system (e.g., the feedback device) may be configured to output the feedback signal in real time. For example, the control device is configured to receive the actual position of the intervention apparatus in real time, to determine the deviation of the actual position from the target position, and to control the output of the feedback signal by the feedback device in real time as a function of the deviation. In one embodiment, the actual position of the intervention apparatus is an actual position of the intervention apparatus (e.g., an intervention apparatus acquired in real time at that moment). In one embodiment, “real time” provides, for example, that any acquisition times for receiving the actual position of the intervention apparatus and any processing times for output of a feedback signal are so small that the user does not perceive any significant delay between an actual change in position of the intervention apparatus and a feedback signal modified accordingly.

The actual position of the intervention apparatus may, for example, be an actual position of the intervention apparatus at that moment. The target position of the intervention apparatus may, for example, be a predetermined target position of the intervention apparatus.

The target position may have been defined before the medical intervention (e.g., planned).

The target position may, for example, include a target, such as a target region, and/or a target point within the patient, of the intervention apparatus and/or an entry point of the intervention apparatus on the surface of the body of a patient. The target position of the intervention apparatus may, for example, include a path of the intervention apparatus. The path may, for example, be a path between a start, such as an entry point of the intervention apparatus on the surface of the body of a patient, as far as a target (e.g., a target region and/or a target point within the patient).

The intervention apparatus may, for example, be a needle (e.g., a surgical needle and/or a biopsy needle).

The use of a non-visual feedback signal, instead of a visual display on the screen in accordance with the prior art, enables the user to concentrate better on the intervention as such. In one embodiment, the system may make a non image-based needle guidance possible.

In one embodiment, the actual position of the intervention apparatus acquired by an imaging device (e.g., by a magnetic resonance device, a computed tomography device, an X-ray device, and/or an ultrasound device) includes acquired position data. In one embodiment, the actual position may be described by such position data.

In one embodiment, the actual position may be determined from image data that is acquired by an imaging device (e.g., by a magnetic resonance device, a computed tomography device, an X-ray device, and/or an ultrasound device). Such image data may be acquired and/or evaluated in real time.

In one embodiment, the feedback signal includes an acoustic signal and/or a haptic signal. In one embodiment, such signals do not divert the attention of the user from the intervention as such or only do so to a slight extent.

The acoustic signal may be a signal able to be transmitted by sound waves. An acoustic signal may be a signal able to be perceived by a hearing organ of the user of the intervention apparatus.

The haptic signal may be a pressure signal acting on the body (e.g., the skin) of the user of the intervention apparatus.

In one embodiment, the feedback signal has a signal pattern that is dependent on the deviation determined.

The signal pattern may be suitable for transmitting to the user information about the deviation determined. The signal pattern, for example, has an encoding, through which messages may be transferred to the user.

In one embodiment, the feedback signal includes an acoustic signal that, depending on the deviation of the actual position from the target position, has a change in pitch and/or a change in volume and/or interruptions of different lengths. In one embodiment, using the change in pitch and/or interruptions of different lengths, information about the deviation of the actual position from the target position may be transferred to the user.

A change in pitch may, for example, include a continuous change in the frequency of the acoustic signal from a start frequency to an end frequency. This may also be referred to as a frequency sweep. A change in pitch may also include jumps from a first frequency to another frequency.

A change in volume may, for example, be a continuous change in the amplitude of the acoustic signal from a start amplitude to an end amplitude. This may also be referred to as an amplitude sweep. A change in volume may also include jumps from a first amplitude to another amplitude.

In one embodiment, the feedback signal includes a haptic signal that, depending on the deviation, has a change in pressure and/or interruptions of different lengths.

In one embodiment, the feedback signal includes a number of part feedback signals able to be distinguished by the user of the intervention apparatus. In one embodiment, this enables more information to be transmitted at the same time to the user. The feedback signal may thus include a number of part feedback signals able to be perceived at the same time by the user of the intervention apparatus.

In one embodiment, the feedback device includes a set of headphones with a first sound transducer and a second sound transducer, where a first of the number of part feedback signals may be output by the first sound transducer and a second of the number of part feedback signals may be output by the second sound transducer.

For example, the first sound transducer may be able to be positioned on the right ear of the user, so that the first of the number of part feedback signals is able to be perceived by the right ear of the user. Correspondingly, for example, the second sound transducer may be able to be positioned on the left ear of the user, so that the second of the number of part feedback signals is able to be perceived by the left ear of the user.

In one embodiment, the first and the second part feedback signals are distinguished such that information about the actual position of the intervention apparatus is able to be derived for the user from the difference.

In one embodiment, the deviation of the actual position from the target position is able to be described by a number of deviation coordinates, where the number of part feedback signals is dependent on values of the number of deviation coordinates.

The deviation coordinates are coordinates of an N-dimensional coordinate system, for example (e.g., a two- or three-dimensional coordinate system). The orientation of such a coordinate system may be defined, for example, as a function of the target position. For example, the orientation of such a coordinate system may be defined as a function of a path that describes the target position of the intervention apparatus.

For example, the coordinate system may include coordinate axes that span a plane, in which at least a part of the path (e.g., the target of the path) is located.

For example, the coordinate system may include a coordinate axis that runs through the target of the intervention apparatus and/or an entry point of the intervention apparatus.

In one embodiment, the deviation of the actual position from the target position is able to be described by a number of deviation coordinates, where each of the number of part feedback signals is assigned one of the number of deviation coordinates. In one embodiment, the feedback signal is dependent on the number of deviation coordinates. For example, each part feedback signal is dependent on the deviation coordinate assigned to the respective part feedback signal.

A medical imaging device that includes a system described above for supporting a medical intervention is further provided.

The medical imaging device may, for example, include a magnetic resonance device, a computed tomography device, an X-ray device, and/or an ultrasound device

In one embodiment, the medical imaging device is configured to acquire an actual position of the intervention apparatus.

Further, a method for output of a feedback signal is provided. The advantages of the method essentially correspond to the advantages of the system for supporting a medical intervention, which have been given above in detail. Features, advantages, or alternate forms of embodiment mentioned here may also be transferred to the method and vice versa. For example, the subject matter of the claims may also be developed with the features that are described or claimed in conjunction with a method.

The method for output of a feedback signal includes receipt of an actual position of an intervention apparatus and a target position of the intervention apparatus by a control device, determination of a deviation of the actual position from the target position by the control device, and an output of a non-visual feedback signal by a feedback device with the aid of the deviation determined.

For example, position data of an intervention apparatus may be acquired by a medical imaging device. The target position of the intervention apparatus may, for example, be determined in advance.

In one embodiment, the method includes a definition of the target position of the intervention apparatus, where the definition of the target position of the intervention apparatus includes a definition of a target (e.g., of a target region). The target region may, for example, be a spatial region that is located up to a defined distance around a target point.

In one embodiment, the deviation of the position data from the target position is described by at least one deviation coordinate, where the non-visual feedback signal is an acoustic feedback signal. The acoustic feedback signal has a change in pitch (e.g., over time), where the change in pitch is determined as a function of the at least one deviation coordinate.

In one embodiment, the determination of the deviation of the actual position from the target position includes a determination of a target plane that at least partly includes the target, a calculation of a projection point by projection of the intervention apparatus (e.g., as a part of the intervention apparatus) onto the target plane with the aid of the actual position of the intervention apparatus, and a calculation of the deviation of the projection point from the target (e.g., from the target region) in the target plane.

The intervention apparatus may be a needle, for example, and the projected part of the intervention apparatus may be a tip of the needle, for example.

The target plane may be a plane that runs in parallel to a surface and/or to a tangential plane of the surface of a patient. For example, the target plane may run in a point tangential to the surface of a patient, at the point at which the intervention apparatus enters the patient.

In one embodiment, the intervention apparatus is a needle (e.g., a straight needle), and the projection of the needle occurs in an extension of the needle (e.g., through the tip of the needle).

In one embodiment, the deviation of the projection point from the target (e.g., from the target region) in the target plane is described by a first deviation coordinate of a first coordinate axis and a second deviation coordinate of a second coordinate axis. The first coordinate axis and the second coordinate axis are not oriented in parallel, but at right angles to one another, for example. The first coordinate axis and the second coordinate axis are oriented in parallel to the target plane. The output of the non-visual feedback signal includes the output of a first feedback signal and the output of a second feedback signal. The first feedback signal is created as a function of the first deviation coordinate and/or the second deviation coordinate. The second feedback signal is created as a function of the first deviation coordinate and/or the second deviation coordinate.

In one embodiment, the determination of the deviation of the position data from the target position includes a determination of a distance between the intervention apparatus and the target plane. The non-visual feedback signal is output as a function of the distance between the intervention apparatus and the target (e.g., the target region). The distance between the intervention apparatus and the target plane may also be seen as a third deviation coordinate.

In one embodiment, the non-visual feedback signal has interruptions (e.g., interruptions over time), where the interruptions are determined as a function of the distance between the intervention apparatus and the target plane. For example, the non-visual feedback signal is an acoustic feedback signal, where there is silence in the interruptions.

A computer program product is also provided. The computer program product includes a program and is able to be loaded directly into a memory of a programmable control device and has program means (e.g., libraries and auxiliary functions) for carrying out a method of one or more of the present embodiments when the computer program product is executed in the control device. The computer program product in this case may be software with a source code that still has to be compiled and linked or just has to be interpreted, or may include an executable software code that just has to be loaded into the control device for execution.

The computer program product enables the method of one or more of the present embodiments to be carried out quickly, with identical repetitions, and robustly. The computer program product is configured so that the computer program product may execute the method acts by means of the control device.

The control device includes, for example, a working memory, a graphics card, a processor, and/or a logic unit, so that the respective method acts may be carried out efficiently.

The computer program product is stored on a computer-readable medium (e.g., a non-transitory computer-readable storage medium), for example, or is held on a network or server, from where the computer program product may be loaded into the processor of a local control device.

Further, control information of the computer program product may be stored on an electronically-readable data medium (e.g., a non-transitory computer-readable storage medium). The control information of the electronically-readable data medium may be configured such that the control information carries out a method of one or more of the present embodiments when the data medium is used in a control device.

Examples of electronically-readable data media are a DVD, a magnetic tape, or a USB stick, on which electronically-readable control information (e.g., software) is stored. When this control information is read from the data medium and stored in a control device, all forms of embodiment of the method described above may be carried out. In this way, the present embodiments may also be based on the computer-readable medium and/or the electronically-readable data medium.

Further advantages, features, and details of the present embodiments emerge from the exemplary embodiments described below as well as with the aid of the drawings. Parts that correspond to one another are labeled with the same reference characters in all figures.

### BRIEF DESCRIPTION OF THE DRAWINGS

FIG. 1 shows one embodiment of a medical imaging device with a system for supporting a medical intervention;

FIG. 2 shows an exemplary spatial representation of an actual position and a target position of an intervention apparatus;

FIGS. 3-10 show various exemplary signal patterns as a function of a deviation of the actual position from the target position; and

FIG. 11 shows a flow diagram of one embodiment of a method for output of a feedback signal.

### DETAILED DESCRIPTION

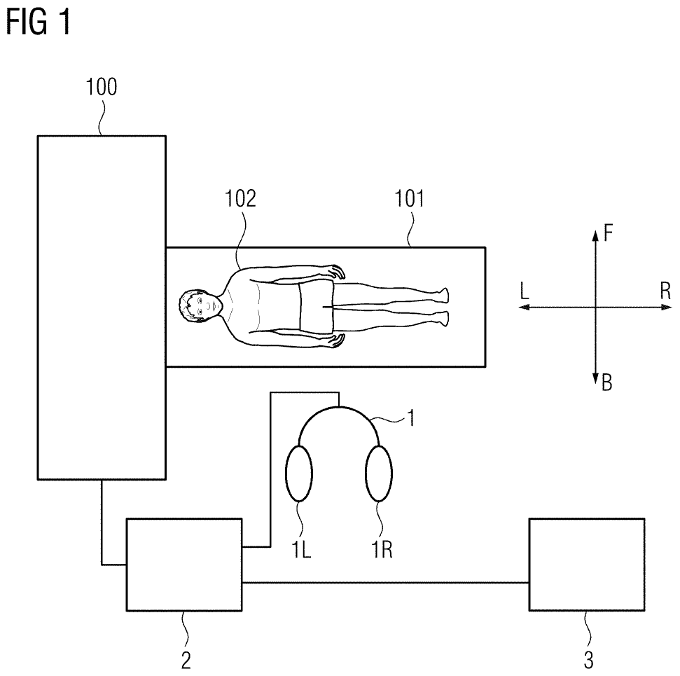

FIG. 1 shows a schematic of one embodiment of a system for supporting a medical intervention including a feedback device 1 for output of a feedback signal to a user of an intervention apparatus shown in the following figures, and a control device 2 for controlling the feedback device.

The directions F (forwards), B (backwards), L (to the left) and R (to the right) correspond to the directions shown in the following figures. In such cases, it is assumed in this example that a medical imaging device (e.g., a magnetic resonance device 100) is located on the left of the user. Located in front of the user is a patient table 101, on which a patient 102 is supported. Other orientations may also be provided.

The control device 2 is configured to receive an actual position of the intervention apparatus, to receive a target position of the intervention apparatus, and to determine a deviation of the actual position from the target position. The actual position is determined with, for example, the aid of the magnetic resonance device 100. The magnetic resonance device 100 records magnetic resonance images of the patient 102 while the intervention apparatus is at least partly within the patient 102.

The target position (e.g., a target path) of the intervention apparatus may be defined by the user, for example, with the aid of a user interface 3 (e.g., planned). The user interface may, for example, have a display unit (e.g., a screen) and an input unit (e.g., a keyboard), by which information and/or parameters may be entered.

Depending on the deviation, the control device 2 is configured to control the output of the feedback signal by the feedback device 1. In this case, the feedback signal is a non-visual feedback signal. In the case shown, the feedback device includes a set of headphones 1, through which acoustic signals may be output as a feedback signal. The set of headphones 1 includes a left-hand sound transducer 1L and a right-hand sound transducer 1R, via which, in each case, different part feedback signals may be output. For example, a first part feedback signal may be output by the left-hand sound transducer 1L, and a second part feedback signal may be output by the right-hand sound transducer 1R. These signals may accordingly be heard by the left ear or right ear of the user.

In one embodiment, an apparatus may be attached to the user as a feedback device, which creates for the user signals able to be perceived haptically. For example, the feedback device may include cuffs that are arranged on the left leg and the right leg of the user in order to apply pressure to the body of the user as a feedback signal.

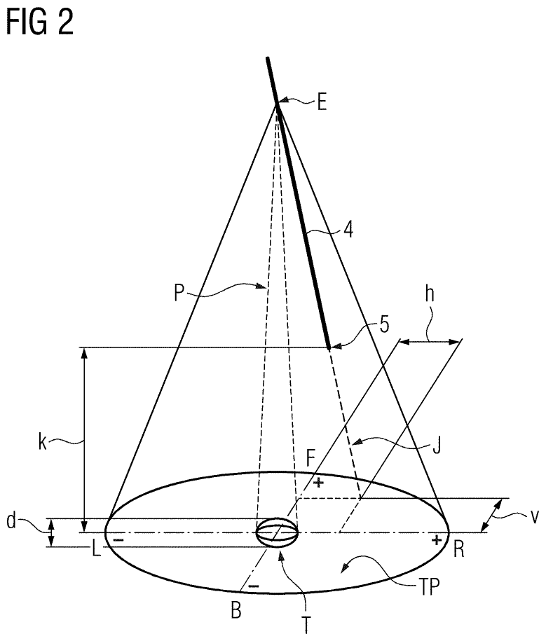

In FIG. 2, a spatial representation of the actual position of an intervention apparatus is shown in the form of a needle 4 and a relative location of the needle 4 in relation to a predetermined path P as target position. The path P is defined by a needle entry point E and a target. The target is described by a target point that lies in a target plane TP. An elliptical target region T with a height d is located around the target point. It may be necessary for the point of the needle 5 to be moved exactly to a target point, but it is sufficient for the point of the needle 5 to be moved within a certain tolerance in the vicinity of the target point (e.g., into the target region T). At the target, the user wishes to carry out a biopsy or a treatment, for example.

The path P may be defined as a straight line between the needle entry point E and a target. The needle entry point E is marked, for example, by a marking at a point on the skin of the patient 102 at which the needle penetrates into the body of the patient 102.

The needle shown in FIG. 2 is located partly within the body of the patient 102 and is now to be brought with tip 5 of the needle to the target. The position of the needle deviates, for example, from the path P and would not reach the target if the needle were to continue to be guided in a current direction. The deviation of the projection J of the needle 4 (e.g., the extension of the needle 4) on the target plane TP is shown in two directions: along the horizontal axis in direction L or R as value h and along the vertical axis in direction B or

F as value v. The values h and v thus represent deviation coordinates that describe a deviation of the actual position from the target position.

The distance between the tip of the needle 5 and the target plane TP is represented by the value k. This provides that the value represents a deviation coordinate that describes a deviation of the actual position from the target position.

In order to guide the tip of the needle 5 to the target, the values of the deviation coordinates are thus to be reduced, so that the tip of the needle 5 arrives in the target region T.

During the intervention, the user hears a part feedback signal through the set of headphones 1 in the right ear and the left ear of the user. The perception of the part feedback signals through the right ear and the left ear enables the user to distinguish the part feedback signals from one another.

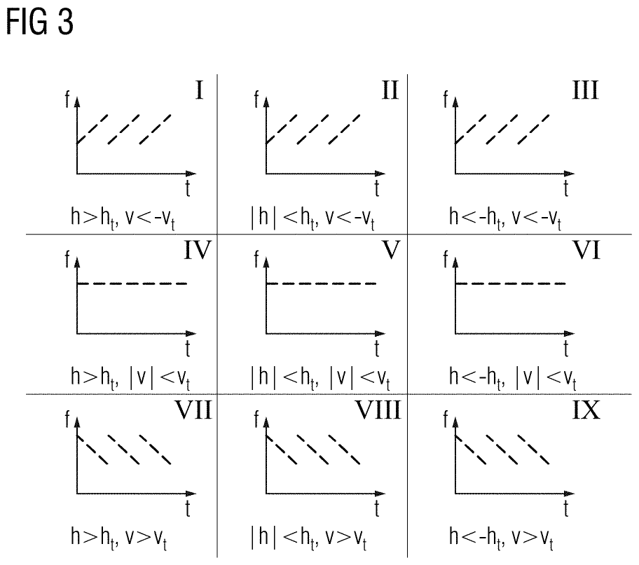

FIG. 3 shows a possible way in which the part feedback signals may be output. Depending on a necessary correction of the actual position of the intervention apparatus, suitable part feedback signals are output via the left-hand sound transducer 1L and/or the right-hand sound transducer 1R. Nine different cases I to IX are shown, which may occur while the needle 4 is being guided to the target region T. The cases represent different signal patterns of the feedback signal that are dependent on the deviation determined.

The deviation of the actual position from the target position in the direction L or R is, for example, able to be described by the deviation coordinate h. The part feedback signals able to be output via the left-hand sound transducer 1L and the right-hand sound transducer 1R are dependent on the deviation coordinate h, as will be explained below.

In the cases in the left-hand column (e.g., in the cases I, IV and VII), a, for example, first part feedback signal is only output by the left-hand sound transducer 1L, (e.g., no second part feedback signal is output by the right-hand sound transducer). These are the cases in which the value h lies above a predetermined threshold value hₜ.

In a similar way, in the cases in the right-hand column (e.g., in the cases III, VI and IX), a part feedback signal is only output by the right-hand sound transducer 1R. These are the cases in which the value h lies below a predetermined threshold value −hₜ.

In the cases in the center column (e.g., in the cases II, V and VIII), a part feedback signal is output in each case both by the left-hand sound transducer 1L and also by the right-hand sound transducer. These are the cases in which the value h lies in a tolerance range between −hₜ and hₜ.

Depending on the value v, the acoustic signal output by the set of headphones 1 has a specific change in pitch, as will be explained below.

In the cases in the top row (e.g., in the cases I, II and III), a sound with a frequency f rising over time is output as a part feedback signal. These are the cases in which the value v lies below a predetermined threshold value −vₜ.

In the cases in the bottom row (e.g., the cases VII, VIII and IX), a sound with a frequency f falling over time is output as the part feedback signal output. These are the cases in which the value v lies above a predetermined threshold value vₜ.

In the cases in the center row (e.g., the cases IV, V and VI), a sound with a frequency f constant over time is output as the part feedback signal. These are the cases in which the value v lies in a tolerance range between −vₜ and vₜ.

An acoustic signal with a frequency f falling over time or a frequency f rising over time is thus output depending on whether the value v lies above a predetermined threshold value vₜ or below a predetermined threshold value −vₜ. If the value v lies in a tolerance range between −vₜ and vₜ, the frequency remains constant. The frequency behavior of the acoustic signal thus gives the user information about the deviation in the directions F or B.

For example, the falling sound in the left ear of case VII provides that the needle 4 would miss a target if the needle 4 were to be moved further without a change in direction, and that instead the needle 4 should be moved to the left (e.g., in direction L because h>hₜ) and backwards (e.g., in direction B because v>vₜ). The fact that the user only hears a signal to the left provides that it is easy and intuitive for the user to grasp that the user should move the needle 4 to the left. The falling sound level also provides that it is easy and intuitive for the user to grasp that the user should move the needle 4 backwards (e.g., towards the user). Similarly, in case I, a correction of the movement should be carried out by the user to the left (e.g., in direction B because v>vₜ) and forwards (e.g., in direction F because v<−vₜ). Further, the increasing sound level makes it easy and intuitive for the user to grasp that the user should move the needle 4 forwards (e.g., away from the user).

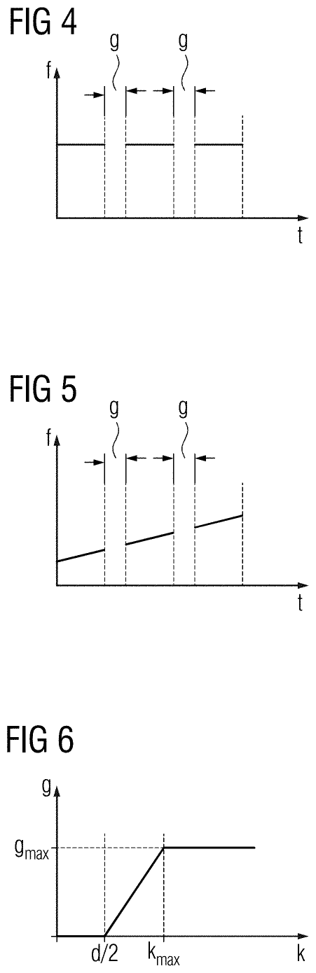

The signal patterns of the part feedback signals shown in FIG. 3 have interruptions, which will be described more closely based on FIGS. 4-6. By way of example, the diagram shown in FIG. 4 represents one of cases IV, V, or VI, and the diagram shown in FIG. 5 represents one of cases I, II, or III. The length g of the interruptions is dependent on distance k of the tip of the needle 5 from the target plane TP, as shown by the diagram of FIG. 6. If the tip of the needle 5 is still at a relatively great distance k>kₘₐₓ from the target plane TP, the length g of the interruption amounts to the value gₘₐₓ. As the distance k becomes smaller between kₘₐₓ and the target region beginning at d/2, the length g of the interruptions becomes ever shorter until the length g becomes equal to zero as from d/2 (e.g., the acoustic signal is then constant). The interruptions are thus determined as a function of the distance k of the intervention apparatus (e.g., of the tip of the needle 5) to target plane TP. It is indicated to the user via the length of the interruptions how far away the tip of the needle 5 still is from the target plane TP.

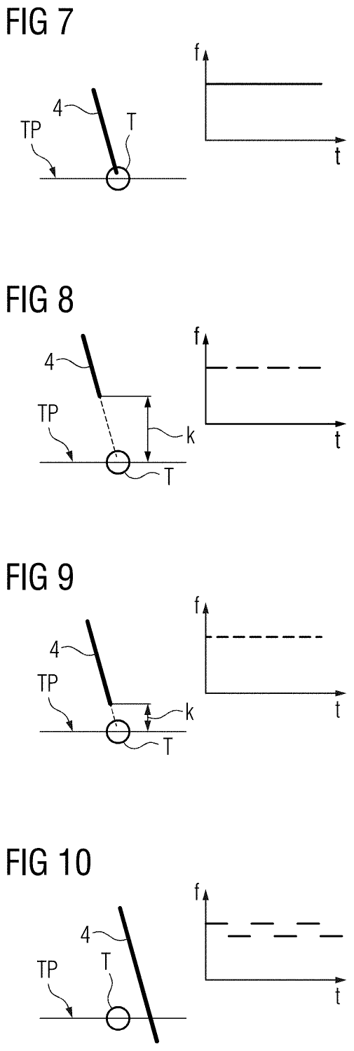

For illustration, a number of scenarios of a medical intervention are shown in FIGS. 7-10, which are supported by the proposed system. In these figures, the position at which the needle 4 is located relative to the target region T and to the target plane TP is shown on the left in each case, and the feedback signal that is output to the user in this situation is shown on the right. For simplification, it is assumed that the tip of the needle 5 is located along the vertical axis (e.g., in direction B or F) within the tolerance range (e.g., |v|<vₜ). Whether the feedback signal shown is output by the left-hand sound transducer 1L, by the right-hand sound transducer 1R, or by both sound transducers 1L, 1R depends on the value h. If, for example, the tip of the needle 5 is located along the horizontal axis (e.g., in direction B or F) within the tolerance range (e.g., |h|<hₜ), the feedback signal is output by both sound transducers 1L, 1R.

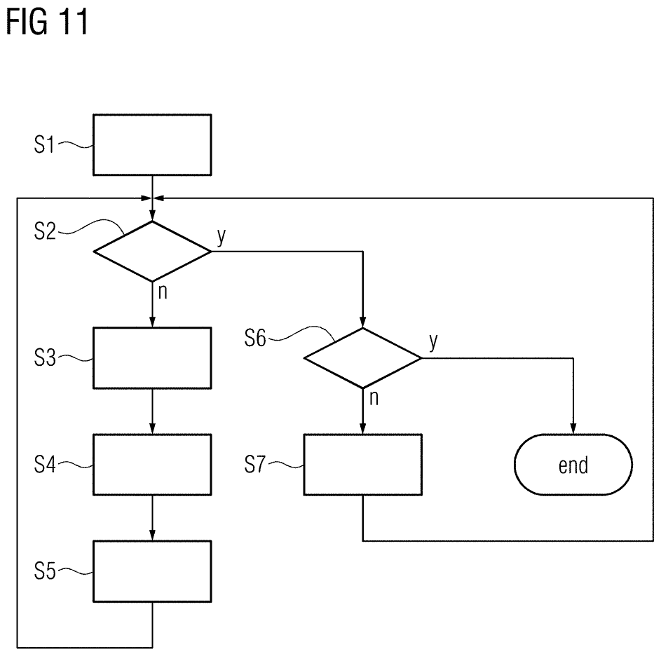

In the scenario shown in FIG. 7, the user has already guided the tip of the needle 5 into the target region T. Thus, the value k<d/2, so that a constant signal is output. In the scenario shown in FIG. 8, the needle is still located in an approach phase to the target region T, so that the value k is still comparatively large. Thus, the interruptions of the signal are still relatively large. The closer the user moves the tip of the needle 5 to the target region T (e.g., the smaller the value k becomes), the shorter the interruptions also become, as is shown in FIG. 9. FIG. 10 shows the case in which the target region T has been missed, so that the tip of the needle is located below the target plane TP. This may be indicated to the user, for example, by the signal jumping backwards and forwards between two frequencies f. The user may then, for example, withdraw the needle 4 a little again in order to bring the tip of the needle 5 back above the target plane TP, so that again, the usual signal patterns, as are shown in FIG. 3, are output, and from there, try again to hit the target region T.

Described in FIG. 11 is a method of how a support of a medical intervention may be carried out.

In act S1, the needle entry point E and the target are defined with a tolerance (e.g., the target region T). Based on this information, a path P is planned as target position of the intervention apparatus. For example, the control device 2 receives the target position of the intervention apparatus. A target plane TP that may be aligned orthogonally to path P is further defined.

In act S2, an actual position of the intervention apparatus (e.g., of the needle 4) is determined in three-dimensional space. For example, the control device 2 receives the actual position of the intervention apparatus. A check is further made as to whether the introduced needle 4 crosses the target plane TP (e.g., whether the target plane TP has a crossing point with the needle 4).

If the needle 4 does not cross the target plane TP, in act S3, based on the actual position of the needle 4, a projection point is determined by projection J of the needle 4 onto the target plane TP.

In act S4, a deviation of the actual position of the intervention apparatus (e.g., of the needle 4) from a target position of the intervention apparatus is determined by the control device 2. The values h, v, and k of the deviation coordinates are determined.

In act S5, depending on the deviation (e.g., of the values h, v, and k), a non-visual feedback signal that includes two part feedback signals, for example, is output by a feedback device (e.g., a set of headphones 1). In the example shown previously, this is done by the sound transducers 1L, 1R.

If the result from act S2 is that the needle 4 crosses the target plane TP, a check is made in act S6 as to whether the tip of the needle 5 is located in the target region T. If so, the method ends. Otherwise, in act S7, a corresponding feedback signal that signals to the user that the user has missed the target is output.

The method described in detail above, as well as the system shown, the control device, and the magnetic resonance device, merely involve exemplary embodiments that may be modified by the person skilled in the art in diverse ways without departing from the scope of the invention. Further, the use of the indefinite article “a” or “an” does not exclude the features concerned also being able to be used multiple times. Likewise, the term “unit” does not exclude the components concerned consisting of a number of interoperating sub-components, which may, if necessary, also be physically distributed.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A system for supporting a medical intervention, the system comprising:
a feedback device configured to output a feedback signal to a user of an intervention apparatus; and
a control device configured to control the feedback device, the control device being configured to:
receive an actual position of the intervention apparatus;
receive a target position of the intervention apparatus;
determine a deviation of the actual position from the target position; and
control the output of the feedback signal by the feedback device as a function of the deviation,
wherein the feedback signal is a non-visual feedback signal.

2. The system of claim 1, wherein the actual position comprises position data acquired by an imaging device.

3. The system of claim 2, wherein the imaging device comprises a magnetic resonance device, a computed tomography device, an X-ray device, an ultrasound device, or any combination thereof.

4. The system of claim 1, wherein the feedback signal comprises an acoustic signal, a haptic signal, or the acoustic signal and the haptic signal.

5. The system of claim 1, wherein the feedback signal has a signal pattern that is dependent on the deviation determined.

6. The system of claim 1, wherein the feedback signal comprises an acoustic signal that, depending on the deviation, has a change in pitch, a change in volume, interruptions of different lengths, or any combination thereof.

7. The system of claim 1, wherein the feedback signal comprises a haptic signal that, depending on the deviation, has a change in pressure, interruptions of different lengths, or a combination thereof.

8. The system of claim 1, wherein the feedback signal comprises a number of part feedback signals that are distinguishable by the user of the intervention apparatus.

9. The system of claim 8, wherein the feedback device comprises a set of headphones with a first sound transducer and a second sound transducer,
wherein a first of the number of part feedback signals is outputable by the first sound transducer, and
wherein a second of the number of part feedback signals is outputable by the second sound transducer.

10. The system of claim 8, wherein the deviation of the actual position from the target position is describable by a number of deviation coordinates, and
wherein the number of part feedback signals is dependent on values of the number of deviation coordinates.

11. A medical imaging device comprising:
a system for supporting a medical intervention, the system comprising:
a feedback device configured to output a feedback signal to a user of an intervention apparatus; and
a control device configured to control the feedback device, the control device being configured to:
receive an actual position of the intervention apparatus;
receive a target position of the intervention apparatus;
determine a deviation of the actual position from the target position; and
control the output of the feedback signal by the feedback device as a function of the deviation,
wherein the feedback signal is a non-visual feedback signal.

12. A method for output of a feedback signal, the method comprising:
receiving, by a control device, an actual position of an intervention apparatus and a target position of the intervention apparatus;
determining, by the control device, a deviation of the actual position from the target position; and
outputting, by a feedback device, a non-visual feedback signal with the aid of the determined deviation.

13. The method of claim 12, further comprising defining the target position of the intervention apparatus,
wherein the definition of the target position of the intervention apparatus comprises a definition of a target.

14. The method of claim 13, wherein the deviation of the position data from the target position is described by at least one deviation coordinate,
wherein the non-visual feedback signal is an acoustic feedback signal,
wherein the acoustic feedback signal has a change in pitch, and
wherein the change in pitch is determined as a function of the at least one deviation coordinate.

15. The method of claim 13, wherein determining the deviation of the actual position from the target position comprises:
determining a target plane that at least partly comprises the target;
calculating a projection point by projection of the intervention apparatus onto the target plane with the aid of the actual position of the intervention apparatus; and
calculating the deviation of the projection point from the target in the target plane.

16. The method of claim 15, wherein the deviation of the projection point from the target in the target plane is described by a first deviation coordinate of a first coordinate axis and a second deviation coordinate of a second coordinate axis,
wherein the first coordinate axis and the second coordinate axis are not oriented in parallel to one another,
wherein the first coordinate axis and the second coordinate axis are oriented in parallel to the target plane,
wherein the output of the non-visual feedback signal comprises an output of a first part feedback signal and an output of a second part feedback signal,
wherein the first part feedback signal is created as a function of the first deviation coordinate, the second deviation coordinate, or a combination thereof, and
wherein the second part feedback signal is created as a function of the first deviation coordinate, the second deviation coordinate, or a combination thereof.

17. The method of claim 15, wherein determining the deviation of the position data from the target position comprises determining a distance between the intervention apparatus and the target plane, and
wherein the non-visual feedback signal is output as a function of the distance between the intervention apparatus and the target.

18. The method of claim 17, wherein the non-visual feedback signal has interruptions, and
wherein the interruptions are determined as a function of the distance between the intervention apparatus and the target plane.

19. In a non-transitory computer-readable storage medium that stores instructions executable by a control device to output a feedback signal, the instructions comprising:
receiving, by a control device, an actual position of an intervention apparatus and a target position of the intervention apparatus;
determining, by the control device, a deviation of the actual position from the target position; and
outputting, by a feedback device, a non-visual feedback signal with the aid of the determined deviation.
